(19) United States
(12) Patent Application Publication
(10) Pub. No.: US 2019/0030111 A1
(43) Pub. Date: Jan. 31, 2019

(54) DETOXIFYING COMPOSITION FOR ORAL ADMINISTRATION AND METHOD FOR PREPARING SAME

(71) Applicant: Ly Eang HAY, (US)

(72) Inventor: Ly Eang HAY, Phnom Penh (KH)

(21) Appl. No.: 16/070,438

(22) PCT Filed: Jan. 16, 2017
(86) PCT No.: PCT/FR2017/050088

§ 371 Date: Jul. 16, 2018

(30) Foreign Application Priority Data
Jan. 21, 2016 (FR) 16 50470

(51) Int. Cl. A61K 36/81; A61K 9/00; A61K 45/06; A61P 1/16; A61P 1/14; A61K 36/899; A61K 36/9068; A61K 36/63; A61K 36/53; A61K 9/16; A61K 9/48; A61K 9/20; A23L 2/52; A23L 33/105

(52) U.S. Cl. CPC A61K 36/81; A23V 2002/00; A61K 9/0095; A61K 9/0056; A61K 45/06; A61P 1/16; A61P 1/14; A61K 36/899; A61K 36/9068; A61K 36/63; A61K 36/53; A61K 9/1652; A61K 9/485; A61K 9/4858; A61K 9/2009; A61K 9/2013; A61K 9/2054; A23L 2/52; A23L 33/105; A61K 2236/15; A61K 2236/17; A61K 2236/331; A61K 2236/333; A61K 2236/53; A61K 9/0053

(57) ABSTRACT

A detoxifying composition for oral administration includes dried inflorescence stems from the pepper plant, that are optionally ground and optionally sieved, and/or a liquid or dry extract of pepper plant inflorescence stems. The pepper plant inflorescence stems are advantageously chosen from inflorescence stems from the Piper nigrum, or Piper longum pepper plants. The food composition or supplement or fortified foodstuff or dietary product containing same is used in order to promote the elimination of waste from the organism, in particular by the liver.

### FIELD OF THE INVENTION

The present invention relates to the field of detoxifying and removing waste from the body.

More particularly, the present invention relates to a detoxifying composition containing a plant-based active agent.

The present invention also relates to the preparation of said composition and the use thereof for promoting the hepatic removal of waste from the body.

### PRIOR ART

The manufacture, use and release of xenobiotic substances, including synthetic chemical products and metals, have increased exponentially and are found in the environment. Consequently, exposure to these persistent toxic substances which are bioaccumulable in humans has increased. This exposure to toxins has been associated with deleterious effects on health, including endocrine disturbances, neurological effects, effects on reproduction and also cancers and cardiovascular diseases. Among these chemical products associated with diseases and disorders are organochlorine pesticides, phthalates, bisphenol A and polybromodiphenyl ethers.

The human diet contains a wide variety of carcinogenic and anti-carcinogenic natural molecules. One way of identifying potential chemopreventive agents among food constituents is to monitor the variations in the levels of enzymes of the biotransformation system which can detoxify carcinogenic chemical products. Specifically, the metabolism of xenobiotics and endobiotic compounds involves enzymatic systems leading to products that have been rendered inactive, enabling their removal from the body.

The term “detoxification” has become common in modern society and is generally associated with weight loss, recuperation from an addiction, or a panacea for numerous unspecified adverse conditions. In the present patent application, the terms “detoxification” or “detoxifying” follow the scientific definition which is much more precise and concerns the process of eliminating poisons or toxins or the effects thereof from an area or an individual, and also the conversion of toxic products into nontoxic substances or any reaction, optionally enzyme-catalyzed, for consuming toxic metabolites without causing damage.

This conversion may especially be performed by the liver, according to two well-established pathways for the chemical modification of “toxic” compounds. These two hepatic pathways are commonly referred to as Phase I and Phase II. Phase I involves the use of a microsomal group of P450 isoenzymes. These mixed-function oxidases generally involve oxidation, reduction, hydrolysis, hydration and isomerization reactions. The toxin is rendered water-soluble by these reactions and thus more readily excreted (i.e. less “toxic” to the body). The enzymes of the Phase I pathway are known to metabolize numerous medicaments (for example phenobarbital), petrochemical hydrocarbons, and also steroid hormones, such as estrogens and testosterone. The Phase II pathway conjugates the metabolites of Phase I, or derived toxins, with more hydrophilic compounds. The Phase II reaction substrates comprise glutathione, glucuronide, glycine, and sulfide, acetyl and methyl groups. If these biotransformed products have a low molecular weight, they are eliminated in the urine. If these final products have a high molecular weight, they are eliminated in the bile.

Traditionally, the hepatic and renal detoxifying properties of plants such as blessed milkthistle (Silybum marianum), meadowsweet (Filipendula ulmaria), black radish (Raphanus sativus var. niger), artichoke (Cynara scolymus), dandelion (Taraxacum officinale), boldo (Peumus boldus) or molecules such as glycine or betaine have been reported.

Data have shown detoxifying properties for pepper and its action on detoxification enzymes (A. Singh, A. R. Rao, Evaluation of the modulatory influence of black pepper (Piper nigrum, L.) on the hepatic detoxification system, Cancer Letters, 72, pages 5-9, 1993; K. Singletary, Black pepper—overview of health benefits, Nutrition Today, Vol. 45, 1, pages 43-47, January/February 2010; I. M. Scott et al., A review of Piper spp (Piperaceae) phytochemistry, insectidical activity and mode of action, Phytochem. Rev., 7, pages 65-75, 2008). Black pepper Piper nigrum L. is the spice most commonly used worldwide, and its extracts have been used in traditional medicine in numerous cultures. The pepper plant is a creeper with a voluble ligneous stem attached to its support by its side branches. The leaves, with an acute oval-shaped limb across which run 3-4 almost parallel veins, are alternate, and the achlamydeous and sessile flowers are grouped into pendulous spikes of 20-30 units. The fruit is a berry 4-8 mm in diameter, which changes from green to red during maturation. The following are conventionally distinguished:
green pepper, which is the fresh whole berry, picked when green;
white pepper, which is the fruit harvested at full maturity. After immersion in water for several days, the fruit are freed of the pericarp and of the outer part of the mesocarp, and then dried;
black pepper, for which the spikes are harvested as soon as the first berries have turned red. After drying, the fruit are separated from the stalks.

Piper nigrum is used for the production of both black pepper (from the unrefined fruit) and white pepper (from the hulled mature berry). In various ancient documents from diverse cultures (India, China), pepper is used, other than as a spice, for medicinal purposes for treating gastrointestinal disorders or epilepsy. The constituents of black pepper comprise fibers, essential oils, piperine, eugenol, lipase and minerals.

1-Piperoylpiperidine, also known as piperine, is the main bioactive component present in both black pepper and white pepper. The scientific data (Singletary, 2010) demonstrate the effects on health of black pepper, in particular in reinforcing digestive functioning. It has also been shown, for example in rats and mice, that black pepper and piperine can stimulate the digestive enzymes, modify the gastric secretions, modify the gastrointestinal transit time of food, and inhibit diarrhea. Black pepper also has an important effect on the enzymatic system which metabolizes medicaments and phytochemical products. Specifically, firstly, it has been shown (Scott, 2008) that piperine inhibits the activities of detoxifying enzymes: arylhydrocarbon hydroxylase (AHH) and 7-ethoxycoumarin deethylase (7ECDE); methylcoumarin demethylase (MOCD); and also the main enzyme for drug metabolization, Cyp3a4. Secondly, piperine induces the Phase I enzymes (cytochrome b5, cytochrome P450) and Phase II enzymes (glutathione S-transferase GST, acid-soluble sulfhydryl —SH, malondialdehyde MDA) and PSMO (polysubstrate monooxygenase). Induction of transcription of the Phase I and II enzyme genes was confirmed. An ethanolic extract of pepper upregulates cytochromes P450 (Cyp6a8, Cyp9b2, Cyp12d1, Cyp6d4, Cyp6d5 and Cyp6w1) with glutathione S-transferase 51 and glutathione S-transferase E7.

These prior art data thus demonstrate the link between piperine and/or Piper nigrum seed/fruit and detoxification.

The present Applicant has found, in its investigation into the use and upgrading of other parts of the pepper plant, that, surprisingly, the pepper plant inflorescence stems, despite having lower contents of piperine, have detoxifying action, in particular hepatic detoxification.

### DESCRIPTION OF THE INVENTION

The present invention thus relates to a detoxifying composition for oral administration, containing a plant-based active agent, characterized in that it comprises dried, optionally ground and optionally screened pepper plant inflorescence stems and/or a liquid or dry extract of pepper plant inflorescence stems.

The pepper plant inflorescence stems are preferably chosen from the inflorescence stems of the pepper plant Piper nigrum or Piper longum, more particularly the inflorescence stems of the pepper plant from the Kampot region of Cambodia. This makes it possible to upgrade the pepper inflorescence, in particular that from Kampot (Cambodia) bearing the label IG Kampot discerned by the Cambodian Committee on Geographical Indications and certified by the international institution (Ecocert). Specifically, pepper specialists throughout the entire world have for several centuries appreciated the pepper (i.e. the berries) from Kampot for its unique taste and its health benefits. Hitherto, the pepper plant inflorescence stem has not been described to have a particular effect.

The extract is advantageously an extract of said inflorescence stems with a polar solvent, such as water, an alcohol or an aqueous-alcohol mixture. Thus, it is very easy to obtain a drinkable detoxifying composition by simple infusion, decoction or maceration in water of pepper plant inflorescence stems.

The term “infusion” means herein pouring water (polar solvent), which is preferably at the boiling point, onto the pepper plant inflorescence stem (for example 2.5 g of dry matter/100 ml of water) and then leaving to infuse for a period ranging from 1 minute to 2 hours. The term “decoction” defines the same operation, the liquid being kept boiling. As regards maceration, this is performed with a cold solvent. Infusion is preferred.

Thus, the composition according to the invention may be in a drinkable form, such as in the form of an infusion, a decoction, a maceration, a flavored drink or any other drinkable form.

The experimental tests performed by the Applicant indicate, for example, that infusion of this dried inflorescence part gives a drink with a specific pleasant and stimulating flavor. Its consumption aids digestion by facilitating salivation and also increases the digestive enzymes, and participates in eliminating toxins from the body. This drink also has relaxing and tonic properties.

The composition according to the invention advantageously contains at least 1% by weight, preferably from 2% to 15% by weight, more preferably from 3% to 10%, of total polyphenols derived from the pepper plant inflorescence stem, which gives it both antioxidant and detoxifying capacities. For example, the tests performed indicate that the inflorescence stem of the pepper plant (Piper nigrum) in powder form comprises about 3% by weight of polyphenols.

The present invention also relates to a food supplement or a fortified food or a dietetic product comprising the composition according to the invention described above in weight proportions of between 0.1% and 85%, preferably between 3% and 65% and more preferably between 20% and 60%.

The term “food supplement” means herein a foodstuff whose purpose is to supplement a normal diet and which constitutes a concentrated source of nutrients or of other substances which have a nutritional or physiological effect, alone or in combination, sold in dose form. The term “dose” means any conventional form: gel capsules, pellets, tablets, vials or sachets.

When the food supplements are in the form of a soft capsule or a gel capsule, the envelope of these soft capsules or of these gel capsules may especially contain animal gelatin such as fish gelatin, glycerol, or a material of plant origin such as a cellulose or starch derivative, or a plant protein.

When the food supplements are in the form of a gel capsule, a tablet or a granule, the mixture of active agents may be attached to a pulverulent support such as silica, cellulose or maltodextrin.

The term “fortified foods” means herein products such as yogurts or drinks to which have been added nutrient substances, into which has been dissolved the powder or the extract of pepper plant inflorescence stem according to the invention.

Foodstuffs which, as a result of their particular composition or of their particular manufacturing process, differ clearly from common consumption foodstuffs, are suitable for an indicated nutritional purpose and are sold as indicating that they meet this purpose are considered as dietetic products intended for particular nutrition.

The food supplement or fortified food or dietetic product according to the invention may also comprise at least one active agent chosen from: vitamins, such as vitamin B6 or C; carotenoids, such as beta-carotene or lutein; mineral trace elements, such as magnesium, calcium or iron; or extracts of rosemary, blessed milkthistle, black radish, meadowsweet, nettle, horsetail, orthosiphon leaves, lemongrass, ginger or jasmine; additional polyphenols and/or anthocyans.

Other pharmaceutically and/or dietetically acceptable agents may be added to the detoxifying infusion, such as fillers, fluidizers, natural extracts, other vitamins, minerals, trace elements, amino acids, fatty acids, anticaking agents, colorants, acidifying agents, thickeners, preserving agents, sweeteners, adjuvants, colorants or flavorings.

The fillers are advantageously chosen from microcrystalline cellulose and potato or corn maltodextrin.

The thickener used is preferably chosen from potato starch, hydroxypropylmethylcellulose, citrus pectin, guar gum, locust bean gum, agar-agar, konjac gum, hydrogenated oils, beeswax, or a mixture thereof.

The fluidizers may be magnesium silicate, magnesium stearate or colloidal silica.

The anticaking agents are those usually used in the food industry, such as magnesium stearate or colloidal silica.

The other vitamins may be chosen, inter alia, from vitamins B1, B2, B3, B5, B8, B9, B12, D and E.

Citric acid may also be used as an acidifying agent in the composition according to the present invention.

The stabilizers used in the manufacture of the food supplement or fortified food or dietetic product according to the present invention are those conventionally used in the food industry, for example sugar and/or cereal derivatives, such as sorbitol.

The sweeteners that may be used are, inter alia, inulin, xylitol, aspartame, glucose syrup, fructo-oligosaccharide syrup, maltitol powder or syrup, acesulfame potassium, fructo-oligosaccharide, sucralose, neohesperidin, steviosides and/or sodium cyclamate.

Fatty acids may also be added to the composition; they may be chosen from omega-3 and/or omega-6 fatty acids or galactolipids. Amino acids, such as tryptophan, leucine, arginine, glycine and/or methionine, may be added.

The presence of flavorings is no longer necessary, since the pepper plant inflorescence stem from Kampot has organoleptic properties which give the composition particular flavors.

The preserving agents are useful so that the detoxifying composition or said food supplement or fortified food or dietetic product is conserved over time. The preserving agents used may be, for example, potassium sorbate, parabens, sodium benzoate, citric acid, benzoic acid, rosemary or ascorbyl palmitate (antioxidant).

All these compounds are in no way limiting, it being possible for pharmaceutically and/or dietetically acceptable agents to be added to the detoxifying composition or to said food supplement or fortified food or dietetic product according to the present invention. The food supplement, the dietetic product or the fortified food may be in the form of coated or uncoated tablets, multilayer tablets, effervescent tablets, sugar-coated tablets, gel capsules, wafer capsules, powders, granules, sticks, pellets, soft capsules, an aqueous solution or suspension in a glass vial, in a stick or a drink, in a dropper bottle, or in a spray.

Advantageously, the food supplement, the dietetic product or the fortified food is administered to a person 1 to 4 times a day, in the dose form comprising from 5 mg to 4 g and preferably from 10 mg to 2 g of said detoxifying composition according to the invention, over a period which may range from 1 day to 3 months.

The present invention also relates to the process for preparing the detoxifying composition as described above: this process comprises at least one of the following steps i) or ii):

i) harvesting, drying, optional grinding, and optional screening, of pepper plant inflorescence stems;

ii) extraction of fresh and/or dried pepper plant inflorescence stems using a polar solvent, such as water, alcohol or an aqueous-alcoholic mixture, followed by filtration of the extract, and optional concentration of the extract, optionally to dryness.

The extraction may be performed according to various protocols:
decoction comprises the boiling of the inflorescence stem in a polar solvent and maintenance of this boiling for a particular period, especially from 1 minute to 2 hours, in particular about 20 minutes, optionally followed by returning to room temperature.
maceration consists in leaving the pepper plant inflorescence stem to soak in the cold polar solvent for a particular period, so as to extract the soluble compounds therefrom. In particular, the maceration step has a duration ranging from 1 hour to 15 days, and preferably 1 day. Most particularly, it is performed at room temperature, i.e. from 10 to 25° C.
infusion, as described above, consists in pouring the polar solvent, which is preferably at the boiling point, onto the pepper plant inflorescence stem, and then leaving to infuse for a period ranging from 1 minute to 2 hours.
digestion comprises heating the pepper plant inflorescence stem in the polar solvent at a temperature below the boiling point: for example, in the case of water, from 10 to 89° C. and in particular from 25 to 70° C.
“percolation” or “leaching” consists in passing the polar solvent through the pepper plant inflorescence stem, which is either fresh or dried beforehand, and ground.
distillation may be performed, for example, by VMHD (pulsed-vacuum microwave hydrodistillation). During this extraction step, the polar solvent is brought to a pressure above atmospheric pressure. In particular, when the solvent is water, it may be in subcritical form, i.e. the water is at a temperature of between 100 and 374° C. and at a pressure of between 1 and 218 bar. Under these conditions, the water is in liquid form.

The concentration of the extract obtained may be performed under vacuum or by evaporation of some or all of the solvent or by bringing said polar solvent to a temperature within a range extending from room temperature (25° C.) to a temperature 300° C. above the boiling point of the solvent used, and preferably at the boiling point of the solvent used. Thus, the polar solvent is at least partly reduced, or even totally removed, especially by evaporation, by sublimation, and preferably under reduced pressure. The term “boiling point of the solvent” means the highest temperature that the solvent can reach before evaporating freely, in gaseous form. This temperature is calculated at atmospheric pressure of 1 atmosphere (101.3 kPa).

The extraction step of the process according to the invention may be in continuous or batch mode, and may comprise a step involving heating, in particular for a time ranging from 1 second to 15 days and especially from 10 seconds to 8 hours.

During an additional step, the extract recovered after extraction may be sterilized, especially by heating with or without pressure and/or by passing through a membrane.

This extract of pepper plant inflorescence stem may also be disinfected, especially by heating with or without pressure or alternatively by means of a disinfectant.

Finally, this extract is advantageously dried. This drying may be performed by freeze-drying and/or by atomization.

This process of extraction with polar solvent thus makes it possible to obtain an extract comprising at least 1% by weight, preferably from 2% to 15% by weight and more preferably from 3% to 10%, of total polyphenols derived from the inflorescence stem (relative to the solids content or relative to the weight of the dried inflorescence stem).

The present invention also relates to the use of the detoxifying composition, or of the food supplement or fortified food or dietetic product as described above, for promoting the removal, especially via the hepatic pathway, of waste from the body.

This detoxifying composition may also be used by any person wishing to detoxify his body (in a slimming diet, or following a medicinal treatment, for example).

The effects are particularly surprising, since:
firstly, piperine is usually extracted from the pepper plant fruit/seed by alcoholic or supercritical CO₂ solvent extraction, whereas the invention relates to infusion, i.e. an aqueous extraction of the active agents from the pepper plant inflorescence stem;
secondly, since the pepper plant inflorescence contains very little piperine (1.00% by weight of dry matter). The results of the analyses conducted by the Institut de Technologie Alimentaire (in Con Tho, Viet Nam) are presented in table 1:

TABLE 1

| | Polyphenols | Piperine |
|---|---|---|
| Kampot pepper berries | 1.58% | 4.01% |
| (dry matter) | | |
| Kampot pepper plant | 2.99% | 1.00% |
| inflorescence stem | | |
| (dry matter) | | |

For these analyses, the polyphenols and the piperidine were extracted from dried pepper plant inflorescence berries or stems, with 96% ethanol according to the method of standard ISO 5564.

The polyphenols were assayed by spectrophotometry at 765 nm according to the Folin-Ciocalteau Micro method.

The piperine was assayed by spectrophotometry at 343 nm according to the method of standard ISO 5564.

The results are expressed as weight percentage of dry matter.

In the light of the literature teaching, it therefore could not be expected that this part of the pepper plant, with a piperine content four times lower than that of the berries, could have a detoxifying effect.

For these two reasons, the plant part used and the extraction process, which differ from that which is conventionally performed and found in the scientific literature, the detoxifying effect of the infusion of Piper nigrum inflorescence stem was not expected.

The invention thus has the advantage of proposing a novel composition with detoxifying action intended for promoting the removal of waste from the body, at least as efficiently as the compositions currently existing on the market.

The present invention will be described in greater detail by means of the implementation examples below, which are intended to illustrate the invention without in any way limiting its scope.

### EXAMPLES

The Kampot pepper plant inflorescence stems used in the examples that follow underwent:
either drying and grinding to obtain coarse granulates used especially for the preparations in “tea” form;
or drying, grinding and screening to obtain a powder with a particle size of between 300 μm and 800 μm approximately, and generally close to 500 μm;
or extraction with a polar solvent to obtain a liquid extract that can be reduced to dryness.

### Example 1

A tea of dried and ground pepper plant inflorescence stem from Kampot Original, according to the infusion process detailed above, and composed solely of dried and ground pepper plant inflorescence stems from Kampot is prepared (2.5 g of dry matter/100 ml of water). This tea may be used as a liver detoxifier in order to help in removing waste from the body.

### Example 2

A tea composed of dried and ground pepper plant inflorescence stem from Kampot and of lemongrass root, which is known for its tonic digestive properties, is prepared (2.5 and 0.5 g of each dry matter, respectively, per 100 ml of water). This tea is used as a digestive and anti-flatulence aid and for combating bloating.

### Example 3

A tea composed of dried and ground pepper plant inflorescence stem from Kampot and of ginger root is prepared (2.5 g of each dry matter/100 ml of water). Ginger is recommended for its beneficial effects on circulation, and may be used as a tonic and an aphrodisiac.

### Example 4

A tea composed of dried and ground pepper plant inflorescence stem from Kampot and of jasmine blossom is prepared (2.5 g of each dry matter/100 ml of water). This jasmine-flavored tea is used as a relaxant, for combating stress and for facilitating sleep.

### Example 5

A tea composed of dried and ground pepper plant inflorescence stem from Kampot and of orthosiphon leaves is prepared (2.5 g of each dry matter/100 ml of water). This mineral-flavored tea is used to aid in slimming and for removing uric acid.

### Example 6

A food supplement or a fortified food in powder form, subsequently packaged in a sachet (for example in stick form), is prepared by mixing the ingredients presented in table 2:

TABLE 2

| Ingredients | Per stick in mg |
|---|---|
| Pepper plant inflorescence | 500.00 |
| stem powder | |
| Fructo-oligosaccharides | 1200.00 |
| (Flavoring) | (10.00) |
| Sweetener | 5.00 |

Said food supplement or fortified food is able to be diluted in a glass of water or alternatively to be ingested directly by man.

### Example 7

The food supplement or the fortified food containing the ingredients presented in table 3 is packaged in a stopper which can be screwed onto a bottle of 300 ml, 1 L or 1.5 L of water.

TABLE 3

| Ingredients | Per pot in mg |
|---|---|
| Pepper plant inflorescence | 700.00 |
| stem powder | |
| Inulin | 1820.00 |
| (Flavoring) | (25.00) |
| Sweetener | 5.00 |
| Colorant | 10.00 |
| Maltodextrin | qs |

In order to use this supplement or this fortified food, it suffices for the user to screw the stopper containing said composition onto a 300 ml, 1 L or 1.5 L bottle containing water, for example. The recommended dose for the individual will then be one stopper per day.

### Example 8

After mixing the ingredients of table 4 and placing in a vial, followed by an autoclave treatment according to the techniques known to those skilled in the art, a complex is obtained composed of an ethanol/water alcoholic extract of dried pepper plant inflorescence stem (2.5 g in 100 ml of an ethanol/water mixture at 70/30 by volume) and of an ethanol/water alcoholic extract of dried Orthosiphon leaves (1 g in 100 ml of an ethanol/water mixture at 70/30 by volume). The extracts are filtered before being placed in the vial.

TABLE 4

| Ingredients | Per vial |
|---|---|
| Extract of pepper plant inflorescence | 2 ml |
| stem | |
| Extract of Orthosiphon leaves | 1 ml |
| Water | 5 ml |

The recommended administration dose is one vial per day to be diluted in a glass of water.

### Example 9

After mixing the ingredients presented in table 5 and extraction of the mixture, according to techniques known to those skilled in the art, a food supplement or a fortified food in the form of extruded granules is obtained.

TABLE 5

| Ingredients | Per pot (in mg) |
|---|---|
| Pepper plant inflorescence | 18 000.00 |
| stem powder | |
| Ginger powder | 14 000.00 |
| Inulin | 128 000.00 |

The recommended daily dose is 2.6 g of granules, measured out, for example, with a teaspoon, to be diluted in a glass of water.

### Example 10

Gel capsules are prepared according to the formulation presented in table 6.

TABLE 6

| Ingredients | Per gel capsule in mg |
|---|---|
| Pepper plant inflorescence stem | 95.00 |
| powder | |
| Lemongrass powder | 65.00 |
| Excipients: | |
| Colloidal silica qs | 1.00 |
| Magnesium stearate qs | 1.00 |

The recommended daily dose is one gel capsule per day.

### Example 11

Tablets are prepared according to the formulation presented in table 7 below.

TABLE 7

| Ingredients | Per tablet in mg |
|---|---|
| Pepper plant inflorescence stem | 400.00 |
| powder | |
| Jasmine powder | 300.00 |
| Excipients: | |
| Microcrystalline cellulose | 95.00 |
| Colloidal silica | 5.00 |
| Magnesium stearate | 14.00 |

The recommended daily dose is one tablet per day.

### Example 12

An aqueous extract of pepper plant inflorescence stem is prepared by percolation of dried stems (2.5 g per 100 ml of water) followed by evaporating the extract to dryness.

An aqueous extract of Orthosiphon leaves (1 g per 100 ml of water) is prepared by percolation of dried leaves, followed by evaporating the extract to dryness.

TABLE 8

| Ingredients | Per bottle in mg |
|---|---|
| Dry extract of pepper plant | 7500.00 |
| inflorescence stem | |
| Dry extract of orthosiphon | 4000.00 |
| leaves | |
| (Flavoring) | (4000.00) |
| Sweetener | 500.00 |
| Citric acid pH <4.5 | 500.00 |
| Potassium sorbate | 500.00 |
| Sodium benzoate | 500.00 |

The food supplement, the dietetic product or the fortified food according to this example is, after homogeneous mixing of the ingredients of table 8, packaged in a bottle with a measuring cup. The recommended daily dose is 25 ml (to be measured out using the measuring cup) and to be diluted in a glass of water.

### Example 13: Test on Mice

The effect of an infusion of pepper plant inflorescence stem powder from Kampot was evaluated on albino Swiss mice. The animals received the infusion (2.5 g of dry matter/100 ml of water) incorporated into their drinking water, at doses of 10% and 20% by volume, for 20 days. The results obtained demonstrate a significant and dose-dependent increase in the body of the content of glutathione S-transferase (GST) and of sulfurated acid (—SH). GST catalyzes the conjugation of glutathione with mutagenic and carcinogenic electrophilic compounds. Furthermore, GST is capable of binding to a wide variety of toxins. The results obtained showed that high levels of cytochromes b5 and P450, phase I enzymes, were also statistically significant and dose-dependent. The content of malondialdehyde (MDA), a lipid peroxidation marker, was lower in the 20% infusion group. This set of results thus suggests an effect of the composition according to the invention in infusion form on induction of the phase I and phase II enzyme detoxification system.

Although the invention has been described in relation with a particular embodiment, it is clear that it is not in any way limited thereto and that it includes all the technical equivalents of the means described and also combinations thereof if they fall within the context of the invention.

1. A detoxifying composition for oral administration, wherein said detoxifying composition comprises dried, optionally ground and optionally screened pepper plant inflorescence stems and/or a liquid or dry extract of pepper plant inflorescence stems.

2. The composition as claimed in claim 1, wherein the pepper plant inflorescence stems are chosen from the inflorescence stems of the pepper plant Piper nigrum or Piper longum.

3. The composition as claimed in either of claim 1, wherein the extract is an extract of said inflorescence stems with a polar solvent.

4. The composition as claimed in claim 1, wherein said composition contains at least 1% by weight.

5. The composition as claimed in claim 1, wherein said composition is in a drinkable form, such as in the form of an infusion, a decoction, a maceration, a flavored drink or any other drinkable form.

6. A food supplement or a fortified food or a dietetic product comprising the composition as claimed in claim 1, in weight proportions of between 0.1% and 85%.

7. The food supplement or fortified food or dietetic product as claimed in claim 6, wherein said food supplement also comprises at least one active agent chosen from: vitamins, such as vitamin B6 or C; carotenoids, such as beta-carotene or lutein; mineral trace elements, such as magnesium, calcium or iron; or extracts of rosemary, blessed milkthistle, black radish, meadowsweet, nettle, horsetail, orthosiphon leaves, lemongrass, ginger or jasmine; additional polyphenols and/or anthocyans.

8. The food supplement or fortified food or dietetic product as claimed in claim 6, wherein said food supplement is in the form of coated or uncoated tablets, multilayer tablets, effervescent tablets, sugar-coated tablets, gel capsules, wafer capsules, powders, granules, sticks, pellets, soft capsules, an aqueous solution or suspension in a glass vial, in a stick or a drink, in a dropper bottle, or in a spray.

9. A process for preparing the composition as claimed in claim 1, wherein said process comprises at least one of the following steps:
i) harvesting, drying, optional grinding, and optional screening, of pepper plant inflorescence stems;
ii) extraction of fresh and/or dried pepper plant inflorescence stems using a polar solvent, such as water, alcohol or an aqueous-alcoholic mixture, followed by filtration of the extract, and optional concentration of the extract, optionally to dryness.

10. A detoxifying composition as claimed in claim 1 for promoting the removal, especially via the hepatic pathway, of waste from the body.

11. The composition as claimed in claim 2, wherein the pepper plant inflorescence stems are the inflorescence stems of the pepper plant from the Kampot region of Cambodia.

12. The composition as claimed in claim 4, wherein said composition contains between 2% to 15% by weight of total polyphenols derived from the inflorescence stem.

13. The composition as claimed in claim 4, wherein said composition contains between 3% to 10% by weight of total polyphenols derived from the inflorescence stem.

14. A food supplement or a fortified food or a dietetic product comprising the composition as claimed in claim 6, in weight proportions of between 3% and 65%.

15. A food supplement or a fortified food or a dietetic product comprising the composition as claimed in claim 6, in weight proportions of between 20% and 60%.

16. A food supplement or fortified food or dietetic product as claimed in claim 6, for promoting the removal, especially via the hepatic pathway, of waste from the body.
